(19) United States
(12) Patent Application Publication
(10) Pub. No.: US 2008/0125777 A1
(43) Pub. Date: May 29, 2008

(54) Vertebral Stabilizer Having Adjustable Rigidity

(75) Inventors: Michael S. Veldman, Memphis, TN (US); Jonathan Dewey, Memphis, TN (US); Christopher M. Patterson, Olive Branch, MS (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/563,594

(22) Filed: Nov. 27, 2006

(51) Int. Cl. A61B 17/58; A61B 17/56; A61B 17/08

(52) U.S. Cl. 606/61; 606/73; 606/103; 606/151

(57) ABSTRACT

A bio-compatible stabilization system includes one or more inserters and a connector for traversing a space between one or more bony structures. The stabilization system is designed to reduce or eliminate stress shielding effects while functioning as a tension band. The elastic properties of the connector can be selected and set on a per-patient basis to allow variance in range of motion and to tailor the connector to the particulars of a patient, i.e., age, gender, weight, height, condition, and the like.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Other treatment methods include spinal stabilization implants whereby a connecting rod or plate (hereinafter “connector”) is secured to a pair of vertebral members spaced from one another.

Conventionally, the connectors have been made of extremely stiff materials such as stainless steel and titanium. Such relatively rigid materials were often used to allow the connector to take on the majority of the stress placed on the spine. Increasingly, however, there has been a desire to use connectors that are less rigid to reduce the incidence of adjacent vertebral degeneration. A number of plastics and polymers have been developed that have been found to be successful in reducing the incidence of vertebral degeneration. As a result, physicians and surgeons when developing a treatment plan must decide between relatively rigid stainless steel or titanium connectors, or relatively flexible plastic connectors. In some circumstances, a single type connector may be satisfactory, but increasingly there is a need for connectors having both rigid and flexible characteristics. Moreover, there is an increasing need to increase the variability of connectors available to physicians in spinal stabilization treatments.

### SUMMARY

In one aspect of the present disclosure, a connector for dynamic spinal stabilization is presented. The connector includes a first end and a second end with an elongated member connected therebetween. The elongated member has an adjustable rigidity.

In another aspect, the present disclosure is directed to a spinal implant that includes a connector with a first section having a first rigidity and with a second section having a second rigidity different from the first rigidity. The spinal implant also includes an inserter designed to engage the connector to position the connector adjacent an anchor securable to a bony structure.

According to another aspect of the present disclosure, a kit for assembling a spinal stabilization rod is disclosed. The kit includes an elongated member having a first rigidity and a shell configured to surround at least a portion of the elongated member. The shell is also designed to have a second rigidity different from the first rigidity. The shell and the elongated member may be assembled to form an integrated spinal stabilization connector.

In yet another aspect of the invention, a surgical method is presented. The surgical method includes implanting a first bone anchor to a first vertebral body and determining a desired rigidity of a connector having a shell. The surgical method further includes inserting a rigidity component into an interior volume of the shell. The rigidity component is selected based on the desired rigidity, and has a rigidity different than that of the shell. One end of the connector is secured to the first bone anchor. A second bone anchor is implanted to a second vertebral body spaced from the first vertebral body. The method further includes securing another end of the connector to the second bone anchor.

These and other aspects, forms, objects, features, and benefits of the present invention will become apparent from the following detailed drawings and descriptions.

### BRIEF DESCRIPTION OF THE DRAWINGS

FIG. 1 is a pictorial representation of a vertebral column with a vertebral stabilizing system according to one embodiment of the present disclosure.

FIG. 2 is an elevation view of a vertebral stabilizing system according to one embodiment of the present disclosure.

FIG. 3A is a cross-sectional view of a straight connector having an internal stiffener according to one embodiment of the present disclosure.

FIG. 3B is a cross-sectional view of a curved connector according to another embodiment of the present disclosure.

FIG. 4A is an elevation view of the stiffener shown in FIG. 3A according to another embodiment of the present disclosure.

FIG. 4B is an end view of the stiffener shown in FIG. 4A.

FIG. 5A is a cross-sectional view of a connector according to yet a further embodiment of the present disclosure.

FIG. 5B is an exploded view of that shown in FIG. 5A.

FIG. 6 is a cross-sectional view of a connector according to another embodiment of the present disclosure.

FIG. 7 is a cross-sectional view of a connector according to another embodiment of the present disclosure.

FIG. 8 is a cross-sectional view of a connector according to another embodiment of the present disclosure.

FIG. 9 is a cross-sectional view of a connector according to yet a further embodiment of the present disclosure.

### DETAILED DESCRIPTION

The present disclosure relates generally to the field of orthopedic surgery, and more particularly to systems and methods for stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

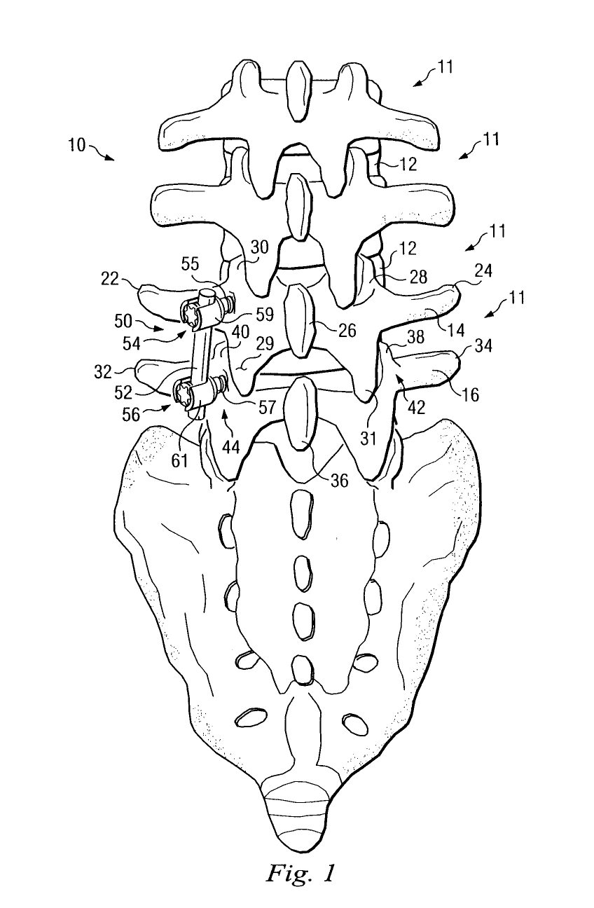

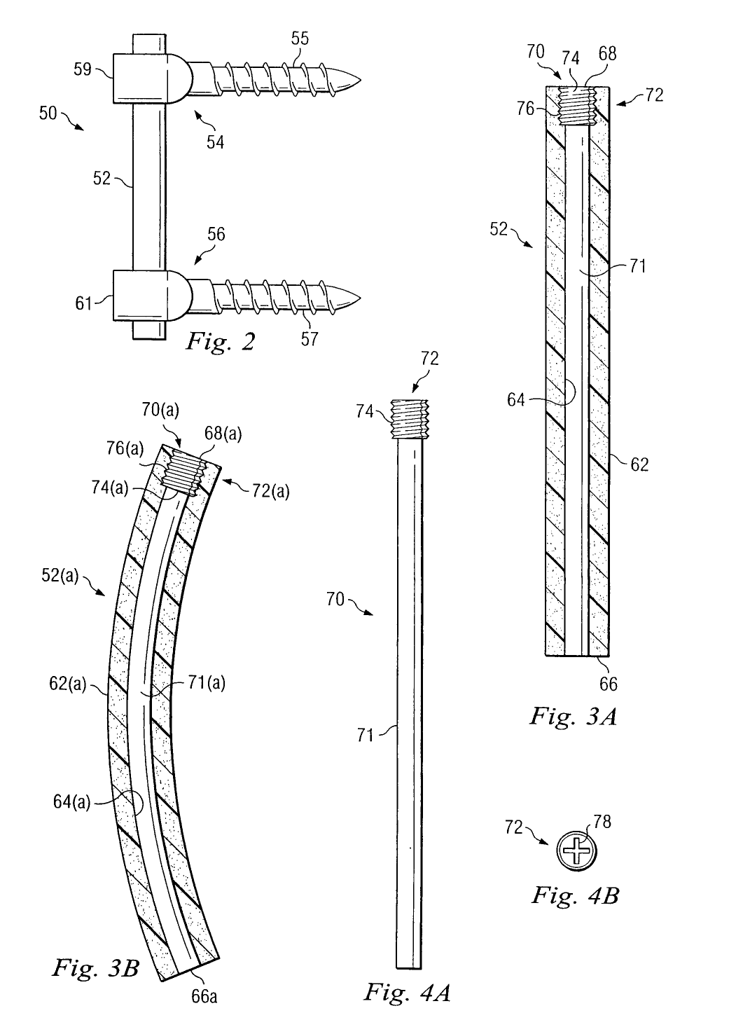

Referring to FIGS. 1-2, the numeral 10 refers to a spinal column having a series of vertebral joints 11, each including an intervertebral disc 12. One of the vertebral joints 11 will be described further with reference to adjacent vertebrae 14, 16. The vertebra 14 includes transverse processes 22, 24, a spinous process 26, superior articular processes 28, 30, and inferior articular processes 29, 31. Similarly, the vertebra 16 includes transverse processes 32, 34, a spinous process 36, superior articular processes 38, 40, and inferior articular processes (not labeled). Although the illustration of FIG. 1 generally depicts the vertebral joint 11 as a lumbar vertebral joint, it is understood that the devices, systems, and methods of this disclosure may also be applied to all regions of the vertebral column, including the cervical and thoracic regions. Furthermore, the devices, systems, and methods of this disclosure may be used in non-spinal orthopedic applications.

A facet joint 42 is formed, in part, by the adjacent articular processes 31, 38. Likewise, another facet joint 44 is formed, in part, by the adjacent articular processes 29, 40. Facet joints also may be referred to as zygapophyseal joints. A healthy facet joint includes a facet capsule extending between the adjacent articular processes. The facet capsule comprises cartilage and synovial fluid to permit the articulating surfaces of the articular processes to remain lubricated and glide over one another. The type of motion permitted by the facet joints is dependent on the region of the vertebral column. For example, in a healthy lumbar region, the facet joints limit rotational motion but permit greater freedom for flexion, extension, and lateral bending motions. By contrast, in a healthy cervical region of the vertebral column, the facet joints permit rotational motion as well as flexion, extension, and lateral bending motions. As the facet joint deteriorates, the facet capsule may become compressed and worn, losing its ability to provide a smooth, lubricated interface between the articular surfaces of the articular processes. This may cause pain and limit motion at the affected joint. Facet joint deterioration may also cause inflammation and enlargement of the facet joint which may, in turn, contribute to spinal stenosis. Removal of an afflicted articular process may result in abnormal motions and loading on the remaining components of the joint. The embodiments described below may be used to stabilize a deteriorated facet joint while still allowing some level of natural motion.

Injury, disease, and deterioration of the intervertebral disc 12 may also cause pain and limit motion. In a healthy intervertebral joint, the intervertebral disc permits rotation, lateral bending, flexion, and extension motions. As the intervertebral joint deteriorates, the intervertebral disc may become compressed, displaced, or herniated, resulting in excess pressure in other areas of the spine, particularly the posterior bony elements of the afflicted vertebrae. This deterioration may lead to spinal stenosis. In one application, the embodiments described below may restore more natural spacing to the posterior bony elements of the vertebrae, decompress an intervertebral disc, and/or may relieve spinal stenosis. Referring still to FIGS. 1-2, in one embodiment, a vertebral stabilizing system 50 may be used to provide support to the vertebrae 14, 16, at least partially decompress the disc 12 and the facet joint 44, and/or relieve stenosis.

Connected at each end to vertebral fasteners 54, 56, a flexible connector 52 may provide compressive support and load distribution, providing relief to the intervertebral disc 12. In addition, the flexible connector 52 may dampen the forces on the intervertebral disc 12 and facet joint 44 during motion such as flexion. Because the flexible connector 52 is securely connected to the vertebral fasteners 54, 56, the flexible connector 52 also provides relief in tension. Accordingly, during bending or in extension, the flexible connector 52 may assist in providing a flexible dampening force to limit the chance of overcompression or overextension when muscles are weak. In addition, the flexible connector 52 allows at least some torsional movement of the vertebra 14 relative to the vertebra 16. In one exemplary embodiment, the fasteners 54, 56 include a pedicle screw 55, 57 that together with anchors 59, 61 secure the flexible connector 52 in place. Such an exemplary fastener is described in U.S. Patent App. Pub. No. 2005/0277922, the disclosure of which is incorporated herein by reference.

Referring now to FIG. 3A, connector 52 is shown in cross-section. As illustrated by the cross-section, the connector 52 has a shell 62 having an inner surface 64 that defines an longitudinal interior chamber (not numbered). The longitudinal chamber is closed at end 66 and open at end 68. The opening at end 68 allows a stiffening rod or member 70 to be inserted into the longitudinal chamber.

As shown in FIG. 4A, the stiffening rod 70 has an elongated shaft 71 extending from a head 72. The head includes a series of threads 74 that engage corresponding threads 76 of the inner surface 64 of the shell 62, FIG. 3A. As will be explained in greater detail below, by using a threaded engagement, as opposed to a adhesive or other sealant engagement, the stiffening rod 70 can be removed from the connector shell 62 to adjust the performance of the connector 52. While a preferred embodiment uses a threaded connection to secure the stiffening rod 70 to the shell 62, it is understood that other types of connections may be used, including, but not limited to the use of adhesives or other sealants, and twist-lock, press fit, and other connections.

As shown in FIG. 4B, head 72 has a tool engagement interface 78 designed to receive a driving tool, such as a screwdriver, for threading the stiffening rod 70 into place. It is understood that one of a number of known tool engagement interfaces could be used.

The connector 52 is constructed such that the material for the shell 62 can have a rigidity or flexibility that is different from that used for the stiffening rod 70. For example, the shell 62 can be fabricated from material that is more flexible than the material used for the stiffening rod 70, or vice-versa. Thus, in one example, the connector has a flexible shell 62 formed of a polymer such as polyetheretherketone (PEEK) whereas the stiffening rod 70 is formed of titanium. Thus, by combining these two materials of different rigidity, the overall flexibility of the connector takes on characteristics of both PEEK and titanium. In other words, the connector 52 is not as stiff as a connector formed completely of titanium or similar material but is not as flexible as a connector formed completely of PEEK or similar material.

Moreover, because the stiffening rod 70 is inserted into the shell 62, the flexibility of the connector can be adapted on a per-patient basis. That is, a surgeon may be supplied a kit of shells of various flexibility and stiffening rods of various rigidity. Based on the particular needs of the patient, the surgeon can then mix-and-match the shells and stiffening rods to construct a connector with a desired flexibility. Furthermore, as the condition of a patient changes, the connector can be surgically accessed, the existing stiffening rod removed, and a replacement stiffening rod inserted to redefine the overall rigidity of the connector.

The connector described with respect to FIGS. 3A, 4A, and 4B has a straight shell 62 and a straight stiffening rod (stiffener) 70. A connector 52(a) having a curved shell 62(a) and a curved stiffening rod 72(a) is illustrated in FIG. 3B. The construction of connector 52(a) is similar to that described with respect to FIGS. 3A, 4A, and 4B, and therefore, for purposes of part illustration, the reference numeral used in FIGS. 3A and 4A have been used identifying the parts of the connector of FIG. 3B with the addition of a parenthetical “a”. Similar to the examples described above with respect to FIGS. 3A, 4A, and 4B, the rigidity of connector 52(a) can be set based on the rigidity of the shell and the rigidity of the stiffening rod.

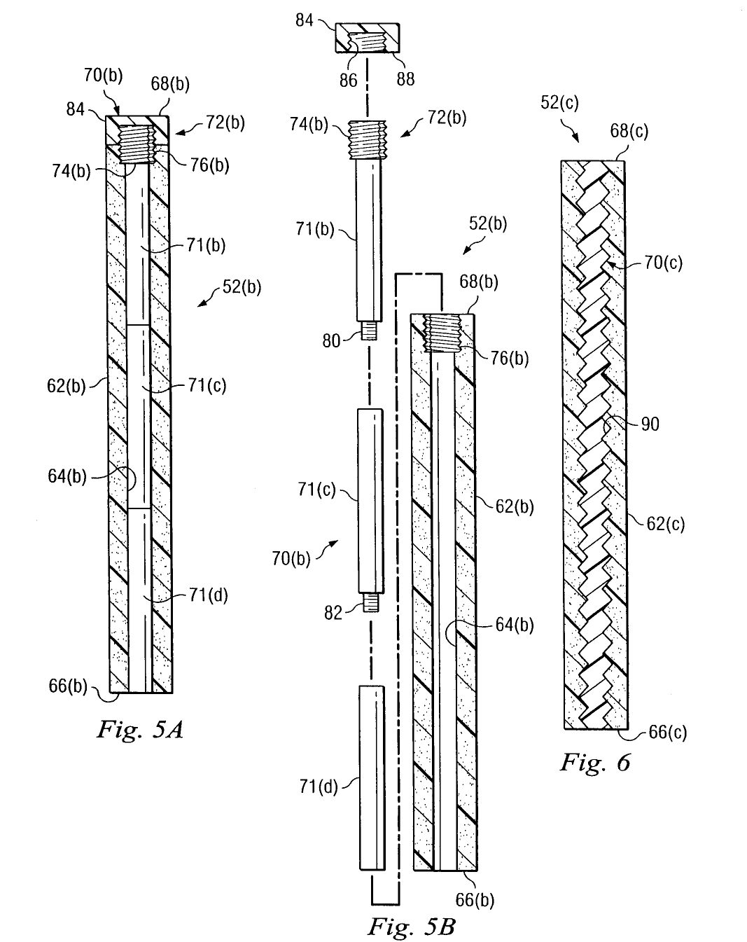

In the connectors illustrated in FIGS. 3A, 3B, and 4A, the stiffening rod runs the entire length of the shell; however, it is contemplated that the stiffening rod may be inserted such that its length is less than the length of the shell. Moreover, as shown in FIGS. 5A and 5B, the shaft of the stiffening rod may be constructed from multiple shaft sections. In this regard, the stiffening rod 70(b) is formed by the threaded engagement of several shaft sections 71(b), 71(c), and 71(d) to one another. To facilitate this threaded engagement, shaft section 71(b) has a threaded stub 80 that is threadingly received by a corresponding socket (not shown) of shaft section 71(c). Similarly, shaft section 71(c) also has a threaded stub 82 that is threaded into corresponding socket (not shown) of shaft section 71(d). When assembled, the stiffening rod 70(b) can then be inserted into the longitudinal chamber of the shell 62(b) as described above. While threaded connectors are shown, it is contemplated that other types of connections could be used, e.g., interference fits.

As the stiffening rod 70(b) is a multi-component structure, shaft sections demonstrating different rigidity characteristics can be assembled to form a single stiffening rod. In this regard, the rigidity characteristics of the stiffening rod can vary along its length. For example, shaft sections 71(b) and 71(d) may be relatively stiff, i.e., composed of titanium, whereas shaft section 71(c) can be relatively flexible, i.e., composed of PEEK. Conversely, in another example, shaft sections 71(b) and 71(d) could be formed of relatively flexible material and shaft section 71(c) could be formed of relatively stiff material.

In the example shown in FIGS. 5A and 5B, the connector 52(b) includes a cap 84 having a threaded interior surface 86. The threaded interior surface 86 threadingly engages threads 74(b) of the head portion 72(b) of the stiffening rod 70(b). In this regard, the length of the head portion 72(b) is such that it extends past the shell 62(b). In one embodiment, an adhesive or other sealant is placed on the under-surface 88 of cap 86 (or on the top surface of the shell) prior to connecting the cap 86 to head portion 72(b). The adhesive further strengthens the connection of the cap 86 to the shell 62(b) and stiffening rod 70(b).

Referring now to FIG. 6, a connector 52(c) according to another embodiment of the present disclosure is shown. Similar to the connectors described above, connector 52(c) has rigidity characteristics that are defined by a relatively flexible outer shell 62(c) and a relatively rigid stiffening rod 70(c). The stiffening rod 70(c) has keys 90 that run along its entire length. The keys 90 are designed to prevent rotation in one or more directions.

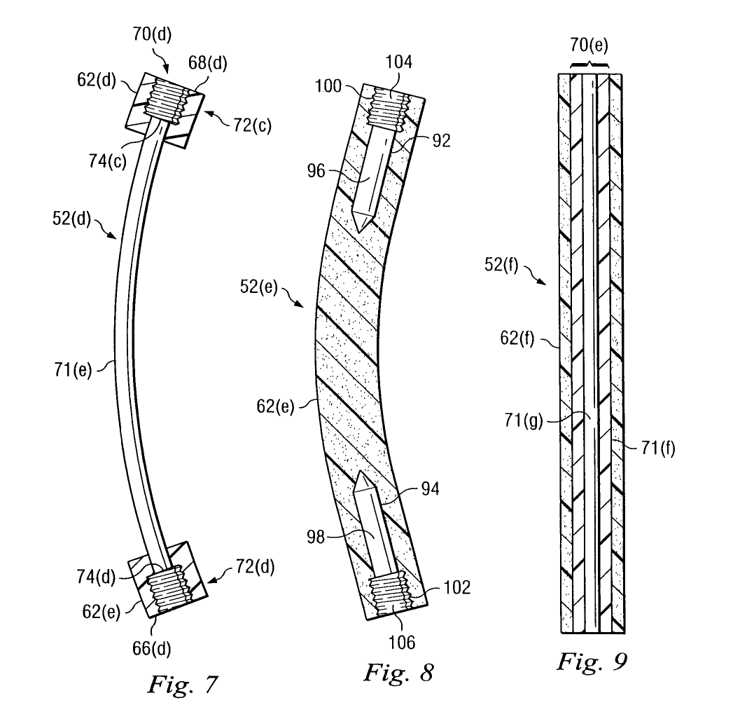

While a number of manufacturing techniques may be used, in one example, connector 52(c) is formed by depositing liquefied stiffening material, such as a gel or other fluid, into the internal chamber of the shell. The stiffening material is then allowed to cure. It is further contemplated that different stiffening materials may be used along the length of the shell. For example, a first liquefied stiffening material may be deposited within the shell, allowed to cure or otherwise harden, and then another stiffening material having a different rigidity than of the first stiffening material is deposited. As such, the rigidity of the stiffening rod 70(c) varies along its length. Also, it is contemplated that the fluids, gels, and the like may be positioned within the shell and allowed to remain in such a fluid or gel-like state to further define the rigidity characteristics of the stiffening rod. Another exemplary manufacturing technique is over-molding whereby the shell is molded around the rod(s) of stiffening material. One skilled in the art will appreciate that other manufacturing techniques may also be used. Moreover, while the diameter of the stiffening rod 70(c) (and the interior chamber of the shell 52(c)) is relatively constant, it is contemplated that the shell may be formed such that the diameter of the stiffening rod varies along its length to further define the overall flexibility of the connector. Another exemplary connector 52(d) is shown in FIG. 7. Connector 52(d) has a relatively thin stiffening rod 70(d) defined by a curved shaft 71(e) connected to threaded ends 72(c) and 72(d). Each threaded end 72(c), 72(d) has a series of threads 74(c), 74(d), respectively. Rather than a single shell that extends along the entire length of the stiffening rod, with connector 52(d), the shell is separated into a pair of sleeves 62(d), 62(e) that threadingly engage threaded ends 74(c), 74(d), respectively. The sleeves 62(d), 62(e) are made of relatively flexible material, e.g., PEEK, whereas the stiffening rod is formed of relatively rigid material, e.g., titanium. Moreover, the sleeves 62(d), 62(e) increase the overall diameter of the ends 66(d), 68(d) of the connector. In other words, because the stiffening rod 70(d) has a relatively smaller diameter, it may be desirable to increase the overall diameter at ends 66(d), 66(e) to improve engagement of the connector in the anchors 54, 56, FIG. 1, which conventionally require a relatively wider connector. Additionally, sleeves 62(d), 62(e) are formed from a relatively flexible material and, as such, the overall flexibility of the connector is defined by flexible components (sleeves) and rigid components (stiffening rod). It is also contemplated that the curved shaft could be formed of relatively flexible material and have one or more stop sleeves (not shown) secured thereto between sleeves 62(d) and 62(e). In this regard, the stop sleeves translate with the shaft during patient movement and prevent full extension of the connector. For example, if a stop sleeve is positioned near sleeve 62(d), the stop sleeve would translate into abutment with sleeve 62(d) during spinal extension. When the stop sleeve abuts sleeve 62(e), the connector will be prevented from further translation thereby preventing over-extension of the spine. Similarly, a stop sleeve could be positioned near sleeve 62(e) in a similar manner to prevent over-flexion of the spine.

Referring now to FIG. 8, a connector 52(e) according to another embodiment of the present disclosure is shown. Connector 52(e) has a relatively flexible shell 62(e) that defines a pair of internal chambers or sockets 92, 94. Each socket 92, 94 is designed to receive a metallic or otherwise relatively rigid insert 96, 98, e.g., screw. Each socket 92, 94 includes a threaded portion 100, 102 designed to engage corresponding threads 104, 106 of inserts 96, 98, respectively. Alternately, each socket could be used to secure the connector 52(e) to threaded studs of other connectors, in a manner similar to that shown in FIG. 5B. In this regard, connector 52(e) could be used as an intermediate component between other rod components connected to one another to collectively form a multi-component connector.

Inserts 96, 98 each have a tool engagement interface (not shown) similar to that illustrated at FIG. 4B. The inserts 96, 98 are designed to not only vary the overall flexibility of the connector 52(e) but are also designed to improve retention of the connector 52(e) in anchors 54, 56, FIG. 1. Moreover, while the inserts are shown as being identical in shape and size, it is understood that the connector 52(e) can be constructed to accommodate different shaped and/or sized inserts to further vary the overall flexibility characteristics of the connector 52(e). For example, the bending moment of the connector 52(e) may favor one end of the connector if dissimilar inserts are used.

FIG. 9 illustrates another exemplary connector 52(f) according to the present disclosure. Connector 52(f) has an outer shell 62(f) and a multilayer stiffening rod 70(e). In the illustrated example, the stiffening rod includes an outer stiffening rod 71(f) with an inner stiffening rod 71(g). The outer stiffening rod 71(f) has a longitudinally extending internal volume sized to receive inner stiffening rod 71(g). While the outer and inner stiffening rods 71(f), 71(g) can be formed of similar materials, it is also contemplated that one of the stiffening rods may be stiffer or more rigid than the other. Similar to the several embodiments describe above, the overall rigidity of the connector 52(f) is defined by the relatively flexible and rigid components 62(f), 71(f), and 71(g). In the illustrated example, interference fits are used to secure the shell 62(f) and the stiffening rods 71(f), 71(g) to one another; however, it is contemplated that threaded, snap-fit, twist-lock, crush-lock, adhesive, thermal (heat) staking, and other types of engagements may be used.

The flexible connectors described herein may be placed directly adjacent the vertebrae, or alternatively, may be spaced from the vertebrae. In some embodiments, placement of the flexible connector directly adjacent the vertebrae may impart specific characteristics to the flexible connector. In some examples, the flexible connector may be spaced from the vertebrae. Accordingly even when the vertebral column is in flexion, causing the spine to bend forward, the first and second vertebral fasteners maintain a line of sight position, so that the flexible connector extends only along a single axis, without bending. In other examples, after placement, the flexible connector may contact portions of the vertebrae during the flexion process. For example, during flexion, the vertebrae may move so that the first and second vertebral fasteners do not have a line of sight position. Accordingly, the flexible connector may be forced to bend around a protruding portion of the vertebrae. This may impart additional characteristics to the flexible connector. For example, because the flexible connector would effectively contact the spinal column at three locations (its two ends and somewhere between the two ends), its resistance to extension might be increased.

In the exemplary embodiments described, the flexible connector is the only component extending from one vertebral fastener to the other. This may be referred to as a single flexible connector. This single flexible connector may be contrasted with conventional systems that employ more than one connector extending between attachment points, such as systems with one component connected at the attachment points and another component extending between attachment points. Because it employs a single flexible connector, the vertebral stabilizing system disclosed herein may be easier and quicker to install, may be less complex, and may be more reliable than prior devices.

Further, the connector is substantially symmetrical such that it may be used on both the left and right sides of the spine. In other embodiments, however, the connector is designed for placement specifically on either the left or right side of the spine. The connector can be tailored for placement on a particular side by changing the general shape, the radius of curvature, the cross-section, or other appropriate features of the connector.

It should be noted however, that a spinal column may employ the flexible connector to extend across a first vertebral space, with a second flexible connector extending across a second vertebral space. Accordingly, more than one vertebral stabilizing system may be used in a spinal column. In some instances where more than one stabilizing system is use, the first and second vertebral spaces may be adjacent. In alternative embodiments, a vertebral stabilizing system may have a single flexible connector with a length allowing it to extend across more than one intervertebral space, with or without connecting to an intermediate vertebra.

In certain anatomies, the vertebral stabilizing system may be used alone to provide decompression or compression to a single targeted facet joint or to relieve pressure on a particular side of the intervertebral disc, such as a herniation area. However, in some instances, a second vertebral stabilizing system may be installed on the opposite lateral side of the vertebrae across from the vertebral stabilizing system. Use of first and second vertebral stabilizing systems may provide more balanced support and equalized stabilization. The second vertebral stabilizing system may be substantially similar to system and therefore will not be described in detail.

The vertebral stabilizing system, as installed, may flexibly restrict over-compression of the vertebrae thereby relieving pressure on the intervertebral disc and the facet joint. In addition, the vertebral stabilizing system may flexibly restrict axial over-extension of the intervertebral disc and the facet joint. By controlling both compression and extension, the vertebral stabilizing system may reduce wear and further degeneration. The flexible connector may also dampen the forces on the intervertebral disc and facet joint during motion such as flexion and extension. Because the flexible connector may be positioned relatively close to the natural axis of flexion, the vertebral stabilizing system may be less likely to induce kyphosis as compared to systems that rely upon inter-spinous process devices to provide compressive and tensile support. Additionally, the system may be installed minimally invasively with less dissection than the inter-spinous process devices of the prior art. Furthermore, an inter-pedicular system can be used on each lateral side of the vertebrae and may provide greater and more balanced stabilization than single inter-spinous process devices.

It should be noted that in some embodiments, the flexible connector may be configured so that orientation in one direction provides one set of stabilizing properties to the vertebrae, while orienting the flexible connector in the other direction would provide a second set of stabilizing properties. In such an embodiment, the body of the flexible member may be asymmetrically shaped.

As described above, the flexible connector can be formed on-the-fly to provide a desired rigidity. The flexible connector can be made of elastic or semi-elastic materials in parts or in its entirety to provide a desired rigidity. The connector can be made of a composite of elastic/semi-elastic and inelastic or rigid materials. Exemplary materials include polyurethane, silicone, silicone-polyurethane, polyolefin rubbers, hydrogels, and the like. The materials can be resorbable, semi-resorbable, or non-resorbable. Exemplary inelastic materials include polymers, such as polyetheretherketone (PEEK), polyetherketoneketone (PEKK), and polylactic acid materials (PLA and PLDLA), metals, such as titanium, NITINOL, and stainless steel, and/or ceramics, such as calcium phosphate and alumina. Further, the various connector components can be solid, hollow, semi-hollow, braided, woven, mesh, porous, or combinations thereof. The connector can also be reinforced or semi-reinforced.

Although disclosed as being used at the posterior areas of the spine, the flexible connector may also be used in the anterior region of the spine to support the anterior column. In such a use, the flexible connector may be oriented adjacent to and connect to the anterior column, and may span a vertebral disc space.

The foregoing embodiments of the stabilization system may be provided individually or in a kit providing a variety of sizes of components as well as a variety of strengths for the connector. It is also contemplated that the connector's characteristics may be color coded or otherwise indicated on the connector itself to expedite identification of a desired connector. It is further contemplated that the connector, or portions thereof, could include radio-opaque markers.

A number of manufacturing techniques are contemplated for making the various connector components described herein. In one embodiment, injection molding is used to form the connector shell. One exemplary injection molding technique is described in U.S. application Ser. No. 11/469,354, the disclosure of which is incorporated herein by reference.

The invention is also embodied in a surgical method for spinal or other bone stabilization. In accordance with this method, a surgeon performs a conventional interbody fusion/nucleus replacement/disc replacement followed by placement of pedicles/bone screws or other inserters into appropriate vertebral or other bony structures. The surgeon may then anchor one end of a connector into a first vertebral or other bony structure. If necessary or otherwise desired, tension is applied to the connector spanning the space between bony structures. Preferably, tension is applied in a limited manner so that inelastic components of the connector are imposing little or no resistance on the applied tension. The un-anchored end of the connector is then anchored to a second vertebral or other bony structure spaced from the first vertebral or other bony structure. Any excess connector extending past the inserters is preferably cut and removed.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. Further, the embodiments of the present disclosure may be adapted to work singly or in combination over multiple spinal levels and vertebral motion segments. Also, though the embodiments have been described with respect to the spine and, more particularly, to vertebral motion segments, the present disclosure has similar application to other motion segments and parts of the body. In the claims, means-plus-function clauses are intended to cover the elements described herein as performing the recited function and not only structural equivalents, but also equivalent elements.

What is claimed is:

1. A connector for dynamic spinal stabilization, the rod comprising:
a first end and a second end; and
an elongated member connected to the first end and the second end, the elongated member having an adjustable rigidity.

2. The connector of claim 1 wherein the elongated member comprises a shell and a stiffening member positioned within the shell.

3. The connector of claim 2 wherein the shell has a first rigidity and the stiffening member has a second rigidity different from the first rigidity.

4. The connector of claim 2 wherein the shell is open at the first end and the stiffening member is configured to be inserted or removed from within the shell by longitudinal translation of the stiffening member through the first end.

5. The connector of claim 2 wherein the elongated member includes multiple stiffening members positioned within the shell.

6. The connector of claim 2 wherein the shell has an inner surface with a first series of threads and the stiffening member has an outer surface with a second series of threads, and wherein the shell and the stiffening member are threadingly engaged to one another.

7. The connector of claim 1 wherein the elongated member is curved.

8. The connector of claim 1 wherein the elongated member comprises an elongated shaft and a first sleeve connected to the shaft at the first end and a second sleeve connected to the shaft at the second end.

9. The connector of claim 8 wherein the first sleeve is threadingly connected to the shaft at the first end and the second end is threadingly connected to the shaft at the second end.

10. A spinal implant comprising:
a connector having a first section having a first rigidity and having a second section having a second rigidity different from the first rigidity; and
an inserter designed to engage the connector to position the connector adjacent an anchor securable to a bony structure.

11. The spinal implant of claim 10 wherein the second section is concentrically disposed within the first section.

12. The spinal implant of claim 11 wherein the first section and the second section are threadingly connected to one another.

13. The spinal implant of claim 10 wherein the connector is curved.

14. The spinal implant of claim 10 wherein the second section is more rigid than the first section.

15. The spinal implant of claim 10 wherein the first section is formed of titanium and the second section is formed of PEEK.

16. A kit for assembling a spinal stabilization rod, the kit comprising:
an elongated member having a first rigidity;
a shell configured to surround at least a portion of the elongated member, the shell having a second rigidity different from the first rigidity; and
wherein the elongated member and shell may be assembled to form an integrated spinal stabilization connector.

17. The kit of claim 16 wherein the shell has a longitudinally extending bore and is configured to slidably receive the elongated member in the longitudinally extending bore.

18. The kit of claim 17 wherein the elongated member has a length less than that of the longitudinally extending bore.

19. The kit of claim 18 wherein the longitudinally extending bore is sized to receive a selected one of multiple elongated members of varying rigidity.

20. The kit of claim 16 wherein the shell has a set of threads engageable with corresponding threads of the elongated member to couple the shell and the elongated member to one another.

21. The kit of claim 16 wherein the shell comprises a first sleeve component and a second sleeve, and wherein the elongated member has a first end and a second end.

22. The kit of claim 21 wherein the first sleeve is connectable to the first end and the second sleeve is connectable to the second end.

23. The kit of claim 16 further comprising another elongated member configured to be internally located within the elongated member, the another elongated member having a third rigidity different from the first rigidity.

24. A surgical method comprising:
implanting a first bone anchor to a first vertebral body;
determining a desired rigidity of a connector having a shell;
inserting a rigidity component into an interior volume of the shell, the rigidity component selected based on the desired rigidity, and having a rigidity different than that of the shell;
securing one end of the connector to the first bone anchor;
implanting a second bone anchor to a second vertebral body spaced from the first vertebral body; and
securing another end of the connector to the second bone anchor.
